(19) United States
(12) Patent Application Publication
(10) Pub. No.: US 2021/0366580 A1
(43) Pub. Date: Nov. 25, 2021

(54) FILTERING ARTIFICIAL INTELLIGENCE DESIGNED MOLECULES FOR LABORATORY TESTING

(71) Applicant: International Business Machines Corporation, Armonk, NY (US)

(72) Inventors: Payel Das, Yorktown Heights, NY (US); Flaviu Cipcigan, Warrington (GB); Kahini Wadhawan, Ferozepur (IN); Inkit Padhi, White Plains, NY (US); Enara C Vijil, Millwood, NY (US); Pin-Yu Chen, White Plains, NY (US); Aleksandra Mojsilovic, New York, NY (US); Tom D.J. Sercu, New York, NY (US); Cicero Nogueira dos Santos, Montclair, NJ (US)

(21) Appl. No.: 16/880,021

(22) Filed: May 21, 2020

(51) Int. Cl. G16C 20/70; G16H 50/20; G16H 70/40; G16H 50/50; G16H 10/40; G16H 70/60; G06F 30/27

(52) U.S. Cl. CPC G16C 20/70; G16H 50/20; G16H 70/40; G06F 30/27; G16H 10/40; G16H 70/60; G16H 50/50

(57) ABSTRACT

Techniques for filtering artificial intelligence (AI)-designed molecules for laboratory testing provided. According to an embodiment, computer implemented method can comprise selecting, by a system operatively coupled to a processor, a first subset of AI-designed molecules from a set of AI-designed molecules as candidate pharmaceutical agents based on classification of the AI-designed molecules using one or more classifiers. The method further comprises selecting, by the system, a second subset of the candidate pharmaceutical agents for wet laboratory testing based on evaluation of molecular interactions between the candidate pharmaceutical agents and one or more biological targets using one or more computer simulations.

### TECHNICAL FIELD

This application relates to artificial intelligence (AI) designed molecules and more particularly to techniques for filtering AI-designed molecules for laboratory testing.

### SUMMARY

The following presents a summary to provide a basic understanding of one or more embodiments of the present disclosure. This summary is not intended to identify key or critical elements or to delineate any scope of the particular embodiments or any scope of the claims. Its sole purpose is to present concepts in a simplified form as a prelude to the more detailed description that is presented later. In one or more embodiments described herein, devices, systems, computer-implemented methods, and/or computer program products are described for filtering AI-designed molecules for laboratory testing.

According to an embodiment, a computer implemented method can comprise selecting, by a system operatively coupled to a processor, a first subset of artificial intelligence (AI)-designed molecules from a set of AI-designed molecules as candidate pharmaceutical agents based on classification of the AI-designed molecules using one or more classifiers. The method further comprises selecting, by the system, a second subset of the candidate pharmaceutical agents for wet laboratory testing based on evaluation of molecular interactions between the candidate pharmaceutical agents and one or more biological targets using one or more computer simulations.

In some implementations, the one or more classifiers comprise one or more neural network or machine learning models that classifies artificial intelligence (AI)-designed molecules as having or not having one or more defined features of a target pharmaceutical agent based on molecular sequences of the AI-designed molecules. With these implementations, first subset can be selected based on the first subset having the one or more defined features. The second subset can further be selected based on the second subset exhibiting one or more target molecular interaction features in the one or more computer simulations.

In one or more embodiments, the candidate pharmaceutical agents can comprise candidate antimicrobial agents. With these embodiments, the classification comprises determining, by the system, whether artificial intelligence (AI)-designed molecules are at least one of: an antimicrobial peptide (AMP), a broad-spectrum antimicrobial, non-toxic, potency or structured. The method can further comprise employing, by the system, the one or more computer simulations to evaluate interaction propensity between the candidate antimicrobial agents and a model lipid bilayer comprising one or more lipids or another cellular component of a pathogen and a forcefield, wherein the selecting the second subset comprises selecting the second subset based on the second subset exhibiting a defined level of the interaction propensity.

In some implementations of these embodiments, the method can further comprise employing, by the system, initial computer simulations to interact test proteins having potent and inactive sequences with a model lipid bilayer comprising one or more lipids or another cellular component of a pathogen and a forcefield, and selecting, by the system, one or more features derived from the model bacterium bilayer that correlate with antimicrobial activity based on the initial computer simulations. The method further comprises evaluating, by the system, the candidate antimicrobial agents for inclusion in the second subset based on whether the candidate antimicrobial agents exhibit the one or more features as determined using the one or more computer simulations.

In various embodiment in which the AI-designed molecules are intended to be antimicrobial agents the wet laboratory testing can comprise at least one of: testing the second subset against one or more gram-positive bacteria or another type of pathogen, testing the second subset against one or more gram-negative bacteria or another type of pathogen, testing a toxicity of the second subset in vitro, or testing a toxicity of the second subset in vivo.

In some embodiments, elements described in connection with the disclosed systems can be embodied in different forms such as a computer system, a computer program product, or another form.

### DESCRIPTION OF THE DRAWINGS

FIG. 1 illustrates a high-level flow diagram of an example pipeline for filtering artificial intelligence (AI)-designed molecular candidates in accordance with one or more embodiments.

FIG. 2 illustrates a block diagram of an example, non-limiting system 200 that facilitates filtering AI-designed molecules for wet laboratory testing in accordance with one or more embodiments.

FIGS. 3A and 3B illustrates block diagrams of example heuristics-based screening components in accordance with one or more embodiments.

FIG. 4 provides a table presenting example heuristics classification results for candidate antimicrobial peptides (AMPs) in accordance with one or more embodiments.

FIGS. 5A and 5B illustrates block diagrams of example simulation-based screening components in accordance with one or more embodiments.

FIG. 6 provides a snapshot of a course-grained molecular dynamics simulation of an AMP in accordance with one or more embodiments.

FIG. 7 provides a table presenting example simulation results for candidate AMPs in accordance with one or more embodiments.

FIG. 8 presents an example confusion matrix in accordance with one or more embodiments.

FIG. 9 illustrates a high-level flow diagram of an example, non-limiting computer-implemented method for filtering AI-designed molecules for laboratory testing in accordance with one or more embodiments.

FIG. 10 illustrates a high-level flow diagram of an example, non-limiting computer-implemented method for filtering candidate AI-designed antimicrobial molecules for laboratory testing in accordance with one or more embodiments.

FIG. 11 provides a table presenting actual simulation results for the top 20 candidate AMPs identified form a set of about 100,000 AI-designed candidate peptides using the disclosed filtering techniques.

FIG. 12 illustrates a block diagram of an example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Technical Field or Summary sections, or in the Detailed Description section.

Machine learning (ML) and artificial intelligence (AI) have has been increasingly used for novel molecule design, particularly with respect to designing novel pharmaceuticals. However, there are many issues when using ML/AI for new pharmaceutical discovery. For example, due to the unbalanced classes and noisy and/or sparse labels, many ML/AI molecule design techniques generate far too many candidates to reasonably evaluate using wet laboratory experiments. For instance, some ML/AI molecule design methods can generate thousands to hundreds of thousands of candidates. Currently, the minimum cost to synthesize and test a single candidate in the wet laboratory environment is between three to five thousand dollars. In addition, the average time to synthesize and test even only 20 candidates in the wet lab is about a month. Accordingly, the development of new pharmaceuticals and other novel molecules using ML and AI is significantly hindered by this highly expensive and time-consuming pipeline.

The disclosed subject matter is directed to systems, computer-implemented methods, and/or computer program products are for efficiently filtering AI-designed molecules for wet laboratory testing. The AI-designed molecules can include various types of pharmaceuticals with the specified properties for a variety of target classes as well as new molecules designed for non-pharmacological uses. The disclosed techniques can be used to significantly decrease the number viable candidates for wet laboratory testing (e.g., from about 100 thousand candidates to about 20 candidates) while also ensuring a relatively high success rate in the wet laboratory testing (e.g., at least a 10% success rate). In one or more embodiments, the filtering process involves a heuristic based screening processes followed by a computer similariton screening process.

In one or more embodiments, the heuristic-based screening process involves developing and/or applying one or more classification models/algorithms (also referred to herein as “classifiers”) to determine or infer whether each (or in some implementations one or more) of the initial candidates has one or more defined target features (i.e., features of interest) based on analysis of their respective molecular sequences (e.g., protein sequence, genetic/nucleotide sequence, polymer sequence, and the like) and/or their chemical structures. The one or more defined target features are selected based on the intended use and/or purpose of the respective candidates and thus can vary. For example, with respect to AI-designed molecules as new pharmaceuticals, the one or more defined target features can be selected based on the desired biological activity of molecules. In this regard, in some embodiments, the candidates can include AI-designed peptides for use as antimicrobial agents. With these embodiments, the one or more defined features can include (but are not limited to), being an antimicrobial peptides (AMPs), being a broad-spectrum antimicrobial, having low or no toxicity, having high potency or not, and having a defined structure (e.g., a secondary structure, such a helix structure, a pleated sheet structure, a coil structure, etc.). In this regard, the one or more classifiers can be used to filter a large initial set of candidate AI-designed molecules to identify smaller subset of candidates that have one or more of the defined features as determined or inferred based on their respective molecular sequences. The subset of candidates selected based on the heuristic-based screening process is generally referred to herein as the “first subset” and can include one or more candidates. The number of candidates included in the first subset can be tailored as appropriate by adapting the filtering criteria (e.g., with respect to number of defined features required, combinations of features required, values indicative of a level of exhibition of the features, values indicative of degree of confidence in the classification inferences, etc.).

The computer simulation screening process evaluates the molecular physics of the candidates included in the first subset using computer simulations to further refine the first subset into an even smaller subset of one or more lead candidates recommended for wet laboratory testing. This smaller subset of candidates is generally referred to herein as the “second subset” of candidates. In various embodiments, the candidates included in the second subset can further be synthesized and evaluated using wet laboratory testing.

In one or more embodiments, the computer simulation process involves using high-throughput computer simulations to simulate the molecular interactions between respective candidates included in the first subset and one or more molecular and/or biological targets (e.g., one or more cellular components of a pathogen). The simulated molecular interactions can be used to identify one or more of the candidates that exhibit one or more behavioral characteristics of interest (i.e., target characteristics). For example, in some embodiments in which the candidates are AMPs, the high-throughput computer simulations can be used to evaluate the candidate peptides included in the first subset to identify and select one or more of these candidates that exhibit consistent interaction propensity with one or more cellular components of a pathogen (e.g., a lipid bilayer and other cellular components).

In some embodiments, training high-throughput computer simulations can be performed for test molecules including test molecules that are known to be effective at achieving the target activity of the AI-designed molecules (e.g., the desired biological activity in implementations in which the AI-designed molecules are pharmaceuticals) and optionally molecules that are known to be ineffective, to identify the one or more behavioral characteristics that correlate with effectiveness in achieving the target activity. These one or more behavioral characteristics can be used as the one or more target characteristics. The computer simulations can then be run on the unknown sequences, that is the sequences of the candidate molecules included in the first subset, to determine whether (and in some implementations to what degree) these candidate molecules exhibit the one or more target characteristics. One or more of those candidate molecules that exhibit a high propensity of the one or more target characteristics can then tested and/or recommended for testing using wet laboratory experimentation.

The disclosed screening techniques were experimentally validated when applied to screen about 100,000 AI-designed AMPs for viable candidates. In this regard, an initial set of 100,000 candidate peptides was reduced to 163 candidate peptides using the disclosed heuristic-based screening process. The 163 candidate peptides were then simulated to test for membrane-binding tendency in accordance with the computer simulation screening process, which resulted in identification of 20 lead candidate peptides that exhibited high and consistent membrane-binding activity in the computer simulations. The 20 lead candidate peptides were then synthesized and tested using wet laboratory experiments for antimicrobial activity and toxicity. Among these 20 lead peptides two final lead AI peptides designed peptides were identified. These two final lead AI-designed peptides among were experimentally validated with strong broad-spectrum anti-microbial activity and low in-vitro and in-vivo toxicity. Both of these novel AMPs were not present in supervised training data used to design the initial candidate peptides. These experiments demonstrate that the disclosed three-stage screening pipeline for AI-generated AMP sequences (e.g., heuristic screening, simulation screening, and wet laboratory screening) yields a success rate of 1 out of 10 at the final stage.

As used herein, the term “AI-designed molecule” is used to refer to a molecule that was designed, generated, or otherwise developed using one or more machine learning (ML) and/or artificial intelligence (AI) techniques. The disclosed AI-designed molecules can include biological molecules (e.g., natural and recombinant peptides, proteins, biopolymers, nucleic acids, polysaccharides, antibodies, hormones, etc.), synthetic molecules, biopharmaceuticals (or “biologics”), and combinations thereof. The disclosed AI-designed molecules can include organic compounds, inorganic compounds, organometallic compounds, or combinations thereof.

The term “peptide” as used herein refers to a polymer of amino acid residues typically ranging in length from 2 to about 50 residues. In certain embodiments the AI-designed peptides disclosed herein range from about 2 to 25 residues in length. In some embodiments the amino acid residues comprising the peptide are “L-form” amino acid residues, however, it is recognized that in various embodiments, “D” amino acids can be incorporated into the peptide. Peptides also include amino acid polymers in which one or more amino acid residues is an artificial chemical analogue of a corresponding naturally occurring amino acid, as well as to naturally occurring amino acid polymers.

As used herein, the term “synthetic” peptide or synthetic AMP is used to refer to a peptide that is chemically synthesized as opposed to host derived. The term “residue” as used herein refers to natural, synthetic, or modified amino acids. Various amino acid analogues include, but are not limited to 2-aminoadipic acid, 3-aminoadipic acid, beta-alanine (beta-aminopropionic acid), 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4 diaminobutyric acid, desmosine, 2,2′-diaminopimelic acid, 2,3-diaminopropionic acid, n-ethylglycine, n-ethylasparagine, hydroxylysine, allo-hydroxylysine, 3-hydroxyproline, 4-hydroxyproline, isodesmosine, allo-isoleucine, n-methylglycine, sarcosine, n-methylisoleucine, 6-n-methyllysine, n-methylvaline, norvaline, norleucine, ornithine, and the like. These modified amino acids are illustrative and not intended to be limiting.

The terms “conventional” and “natural” as applied to peptides herein refer to peptides, constructed only from the naturally-occurring amino acids: Ala, Cys, Asp, Glu, Glu, Phe, Gly, His, Ile, Lys, Leu, Met, Asn, Pro, Gln, Arg, Ser, Thr, Val, Trp, and Tyr. In various embodiments, the disclosed AI-designed peptides comprise only of natural amino acid residues. In some embodiments, the disclosed AI-designed molecules can substitute one or more synthetic or modified amino acids for a corresponding natural amino acid. A compound of the invention “corresponds” to a natural peptide if it elicits a biological activity (e.g., antimicrobial activity) related to the biological activity and/or specificity of the naturally occurring peptide. The elicited activity may be the same as, greater than or less than that of the natural peptide. In general, such a peptide will have an essentially corresponding monomer sequence, where a natural amino acid is replaced by an N-substituted glycine derivative, if the N-substituted glycine derivative resembles the original amino acid in hydrophilicity, hydrophobicity, polarity, etc.

In certain embodiments, AMPs compromising at least 80%, preferably at least 85% or 90%, and more preferably at least 95% or 98% sequence identity with any of the sequences described herein are also contemplated. The terms “identical” or percent “identity,” refer to two or more sequences that are the same or have a specified percentage of amino acid residues that are the same, when compared and aligned for maximum correspondence, as measured using one of the following sequence comparison algorithms or by visual inspection. With respect to the peptides disclosed herein sequence identity is determined over the full length of the peptide. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted using a basic local alignment search tool (BLAST) or the like.

The term “specificity” when used with respect to the antimicrobial activity of a peptide indicates that the peptide preferentially inhibits growth and/or proliferation and/or kills a particular microbial species as compared to other related species. In certain embodiments the preferential inhibition or exterminating is at least 10% greater (e.g., the LD₅₀ being 10% lower), preferably at least 20%, 30%, 40%, or 50%, more preferably at least 2-fold, at least 5-fold, or at least 10-fold greater for the target species.

“Treating” or “treatment” of a condition as used herein may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

The term “high” as used with respect to antimicrobial activity and/or potency is used herein to indicate that the level of antimicrobial activity of an antimicrobial agent (e.g., an AMP or the like) is greater than a defined minimum threshold of antimicrobial activity or potency for a particular bacterial organism. In various embodiments, the minimum threshold can be based on its MIC, its LD₅₀ concentration/or its HC₅₀, concentration, wherein the lower the concentration, the higher the antimicrobial activity and/or potency. For example, in some embodiments, an antimicrobial agent can be considered to have high antimicrobial activity and/or potency if its MIC is less than 250 micrograms per milliliter (μg/mL), more preferably less than 150 μg/mL, more preferably less than 100 μg/mL, more preferably less than 50 μg/mL, and even more preferably less than 30 μg/mL.

The term “low-toxicity” is used herein to indicate any level of toxicity of a pharmacological agent (e.g., including one or more AMPs or another active agent) that is less than defined acceptable threshold of toxicity. In various embodiments, the defined threshold can be based on the MIC of the pharmacological agent relative to its LD₅₀ and/or HC₅₀ concentration. In some implementations, a pharmacological agent (e.g., an AMP or a composition comprising one or more AMPs) can be considered to have low-toxicity if its MIC is less than its LD₅₀ and/or HC₅₀ concentration. In other implementations, a pharmacological agent can be considered to have low-toxicity if its MIC is 60% or less than its LD₅₀ and/or HC₅₀ concentration. In other implementations, a pharmacological agent can be considered to have low-toxicity if its MIC is 50% or less than its LD₅₀ and/or HC₅₀ concentration. In other implementations, a pharmacological agent can be considered to have low-toxicity if its MIC is 30% or less than its LD₅₀ and/or HC₅₀ concentration. In other implementations, a pharmacological agent can be considered to have low-toxicity if its MIC is 25% or less than its LD₅₀ and/or HC₅₀ concentration.

Various embodiments of the disclosed subject matter are exemplified with respect to evaluating AI-designed molecules that are (or are intended to be) new pharmaceuticals, and more particularly to AI-designed AMPs. However, it should be appreciated that the disclosed AI-designed molecule filtering techniques can be used to evaluate a variety of pharmaceuticals with the specified properties for a variety of target classes (e.g., antiviral agents, antineoplastic agents, therapeutic agents, antineoplastic agents, etc.) as well as new molecules designed for non-pharmacological uses. The terms “pharmaceutical”, “pharmaceutical agent”, “medicine”, “medication”, and “bio-active molecule” are used herein interchangeably to refer to a substance that is used (or designed to be used) to diagnose, cure, treat or prevent disease, unless context warrants particular distinctions among the terms.

One or more embodiments are now described with reference to the drawings, wherein like reference numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details. It is noted that the drawings of the present application are provided for illustrative purposes only and, as such, the drawings are not drawn to scale.

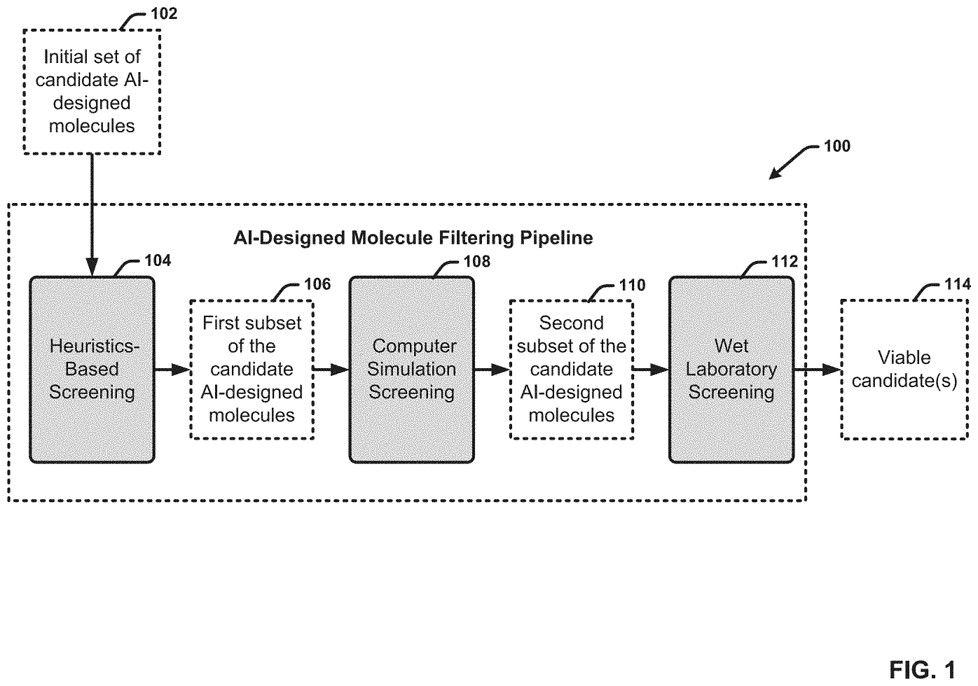

FIG. 1 illustrates a high-level flow diagram of an example pipeline 100 for filtering AI-designed molecular candidates in accordance with one or more embodiments. The pipeline 100 employs a three-phase screening regime to filter an initial set 102 of candidate AI-designed molecules (also referred to herein as “candidate molecules” or simply “candidates”) into one or more viable candidates 114. The three-phases include a heuristics-based screening phase 104, a computer simulation screening phase 108, and a wet laboratory screening phase 112. In accordance with pipeline 100, the heuristics-based screening phase 104 is used to select a first subset 106 of the candidates from the initial set 102 based on one or more predefined target features using one or more classifiers. The computer simulation screening phase 108 is then used to select a second subset 110 of lead candidate AI-designed molecules from the first subset 106 using physics-driven computer simulations to evaluate relevant molecular dynamics of the respective candidates included in the first subset. For example, the computer simulations can simulate molecular interactions between the respective candidates (included in the first subset 106) and one or more molecular/biological targets of the candidate AI-designed molecules (e.g., one or more cellular components of a pathogen). The second subset 110 is then selected based on whether and/or to what degree the candidates exhibit one or more target behavioral characteristics in the computer simulations.

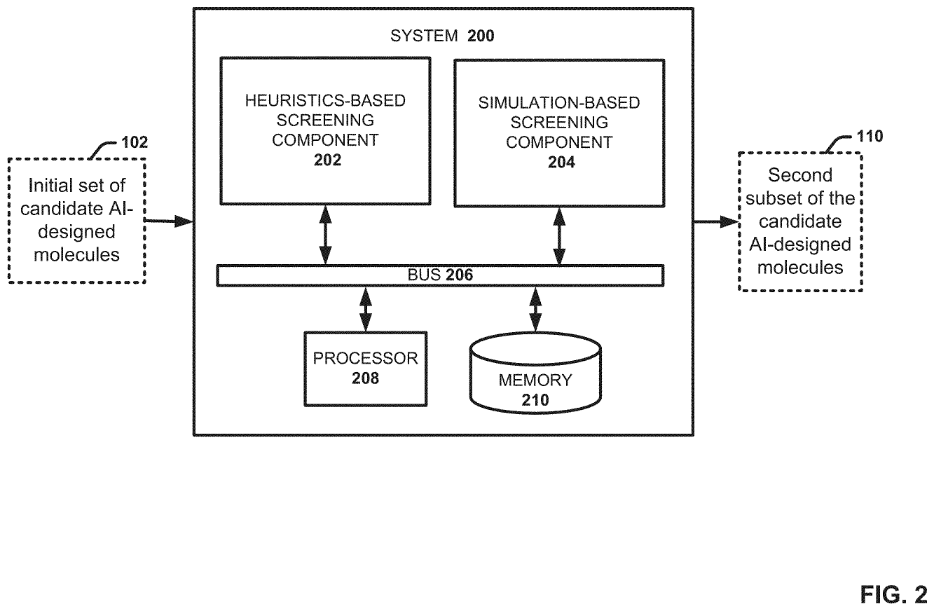

The wet laboratory screening phase 112 can then be used to screen the respective candidates included in the second subset 110 (also referred to herein as the lead candidates) to identify any viable candidates 114. In various embodiments, the wet laboratory screening phase 112 involves synthesizing the lead candidates and performing appropriate in-vitro and/or in-vivo testing to validate whether the lead candidates are viable against one or more pathogens or another molecular target as indicated based on the heuristics-based screening phase 104 and the computer simulation screening phase 108. For example, in one or more embodiments in which the AI-designed molecules include molecules designed to be used as antimicrobial agents (e.g., AMPs), the wet laboratory screening phase 112 can include (but is not limited to) testing the lead candidates against one or more types of gram-positive bacteria and/or gram-negative bacteria or another type of pathogen, and testing the toxicity of the lead candidates in-vitro and/or in-vivo. Additional details regarding the AI-designed molecule filtering pipeline (e.g., pipeline 100) are further described with reference to FIGS. 2-11.

FIG. 2 illustrates a block diagram of an example, non-limiting system 200 that facilitates filtering AI-designed molecules for wet laboratory testing in accordance with one or more embodiments. Embodiments of systems described herein can include one or more machine-executable components embodied within one or more machines (e.g., embodied in one or more computer readable storage mediums associated with one or more machines). Such components, when executed by the one or more machines (e.g., processors, computers, computing devices, virtual machines, etc.) can cause the one or more machines to perform the operations described.

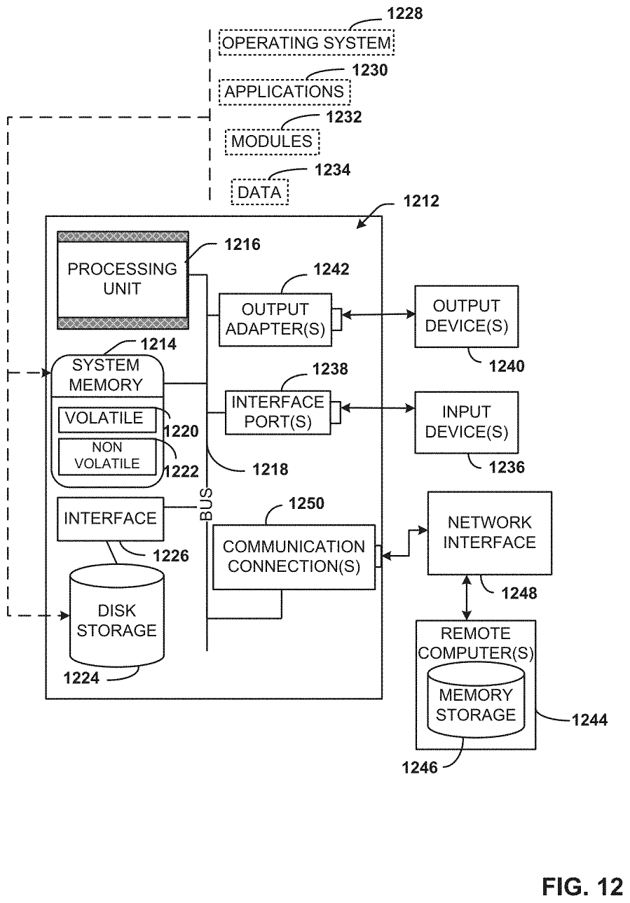

For example, in the embodiment shown, system 200 includes a heuristics-based screening component 202 and a simulation-based screening component 204 that can respectively be or correspond to machine or computer executable components. System 200 can further include or be operatively coupled to at least one memory 210 and at least one processor 208. In various embodiments, the at least one memory 210 can store executable instructions (e.g., the heuristics-based screening component 202, the simulation-based screening component 204, and additional components described herein) that when executed by the at least one processor 208, facilitate performance of operations defined by the executable instructions. System 200 can further include a device bus 206 that communicatively couples the various components of the system 200. Examples of said processor 208 and memory 210, as well as other suitable computer or computing-based elements, can be found with reference to FIG. 12 with respect to processing unit 1216 and system memory 1214, and can be used in connection with implementing one or more of the systems or components shown and described in connection with FIG. 1 or other figures disclosed herein.

In some embodiments, system 200 can be deployed using any type of component, machine, device, facility, apparatus, and/or instrument that comprises a processor and/or can be capable of effective and/or operative communication with a wired and/or wireless network. All such embodiments are envisioned. For example, system 200 can be deployed by, run by, and/or otherwise executed by a server device, a computing device, a general-purpose computer, a special-purpose computer, a tablet computing device, a handheld device, a server class computing machine and/or database, a laptop computer, a notebook computer, a desktop computer, a cellular phone, a smart phone, a consumer appliance and/or instrumentation, an industrial and/or commercial device, a digital assistant, a multimedia Internet enabled phone, a multimedia player, and/or another type of device.

It should be appreciated that the embodiments of the subject disclosure depicted in various figures disclosed herein are for illustration only, and as such, the architecture of such embodiments are not limited to the systems, devices, and/or components depicted therein. In some embodiments, one or more of the components of system 200 can be executed by different computing devices (e.g., including virtual machines) separately or in parallel in accordance with a distributed computing system architecture. System 200 can also comprise various additional computer and/or computing-based elements described herein with reference to operating environment 1200 and FIG. 12. In several embodiments, such computer and/or computing-based elements can be used in connection with implementing one or more of the systems, devices, components, and/or computer-implemented operations shown and described in connection with FIG. 1 or other figures disclosed herein.

In some embodiments, system 200 can be coupled (e.g., communicatively, electrically, operatively, etc.) to one or more external systems, data sources, and/or devices via a data cable (e.g., coaxial cable, High-Definition Multimedia Interface (HDMI), recommended standard (RS) 232, Ethernet cable, etc.). In other embodiments, system 200 can be coupled (e.g., communicatively, electrically, operatively, etc.) to one or more external systems, sources, and/or devices via a network.

According to multiple embodiments, such a network can comprise wired and wireless networks, including, but not limited to, a cellular network, a wide area network (WAN) (e.g., the Internet) or a local area network (LAN). For example, the heuristics-based screening component 202 and/or the simulation-based screening component 204 can communicate with one or more external systems, sources, and/or devices, for instance, computing devices (and vice versa) using virtually any desired wired or wireless technology, including but not limited to: wireless fidelity (Wi-Fi), global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra mobile broadband (UMB), high speed packet access (HSPA), Zigbee and other 802.XX wireless technologies and/or legacy telecommunication technologies, BLUETOOTH®, Session Initiation Protocol (SIP), ZIGBEE®, RF4CE protocol, WirelessHART protocol, 6LoWPAN (IPv6 over Low power Wireless Area Networks), Z-Wave, an ANT, an ultra-wideband (UWB) standard protocol, and/or other proprietary and non-proprietary communication protocols. In such an example, system 200 can thus include hardware (e.g., a central processing unit (CPU), a transceiver, a decoder), software (e.g., a set of threads, a set of processes, software in execution) or a combination of hardware and software that facilitates communicating information between system 200 and external systems, sources, and/or devices.

System 200 facilitates filtering large data sets of AI-designed molecules into a significantly smaller data sets of more targeted and promising candidates (i.e., the second subset of the candidate AI-designed molecules) that are likely to provide the target activity/function for more comprehensive validation experimentation, such as wet laboratory experimentation, clinical trials for new pharmaceuticals, and the like. To facilitate this end, system 200 can include heuristics-based screening component 202 and simulation-based screening component 204.

With reference again to FIG. 1 in view of FIG. 2, the heuristics-based screening component 202 can be configured to perform the heuristics-based screening phase 104 of the pipeline 100 to generate the first subset 106 of the candidate AI-designed molecules and the simulation-based screening component 204 can be configured to perform the computer simulation screening phase 108 of the pipeline 100 to generate the second subset 110 of the candidate AI-designed molecules. As shown in FIG. 1, the output of system 200 includes the second subset 110 of the candidate AI-designed molecules, which correspond to a reduced set of viable candidates that are recommended for additional testing (e.g., wet laboratory testing).

In this regard, system 200 can receive (or otherwise access) an initial set 102 of candidate AI-designed molecules for screening/filtering. The initial set 102 of candidate AI-designed molecules can include any number of candidate molecules (e.g., including hundreds to thousands to hundreds of thousands or more). The type of the AI-designed molecules included in the initial set and/or their target biological and/or chemical activity can vary. In some embodiments, the initial set 102 of candidate AI-designed molecules can include pharmaceuticals designed to provide a specific biological response in association with diagnosing, treating, curing, and/or a particular disease. For example, the initial set 102 of candidates can include AI-designed molecules designed to function as antimicrobial agents, antiviral agents, anti-cancer agents the like. In another more specific embodiment, system 200 can be particularly configured to screen AI-designed peptides designed to function as broad-spectrum antimicrobial peptides. In accordance with this embodiment, the initial set 102 of candidate AI-designed molecules can include a collection of such peptides.

In some embodiments, the initial set 102 of candidate can vary with respect to their molecular sequence and/or chemical structure yet share a common design factor or another common attribute. For example, in some implementations, the initial set 102 of candidates can include molecules that were generated/designed using one or more of the same ML/AI design models. In another example, the initial set of candidates can include molecules that were designed to provide a same or similar target biological/chemical activity or function, and/or target a same or similar biological/molecular target. Additionally, or alternatively, the initial set 102 of candidates can include a collection of AI-designed molecules that vary with respect to one or more of these common factors, randomly sampled AI-designed molecules or the like.

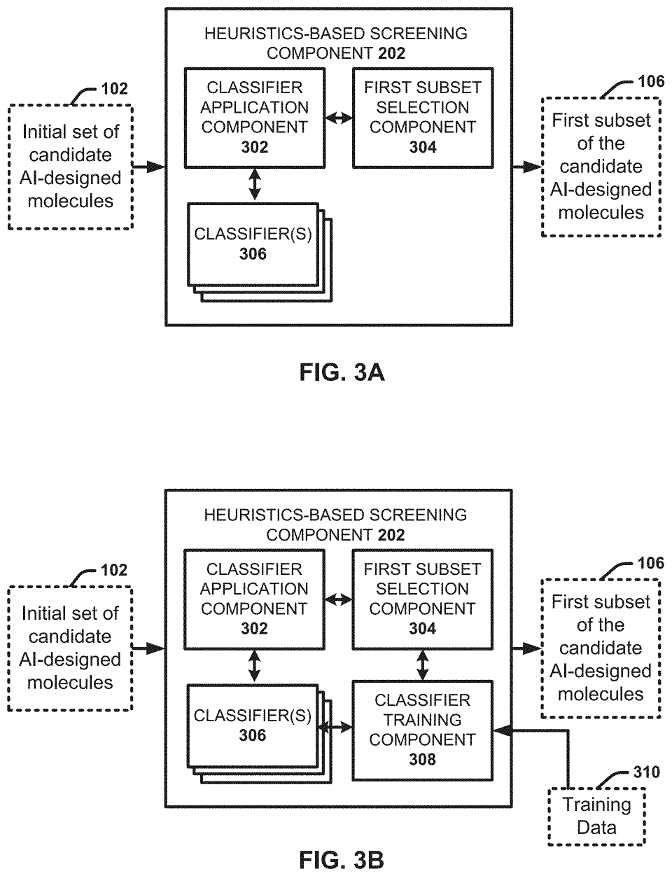

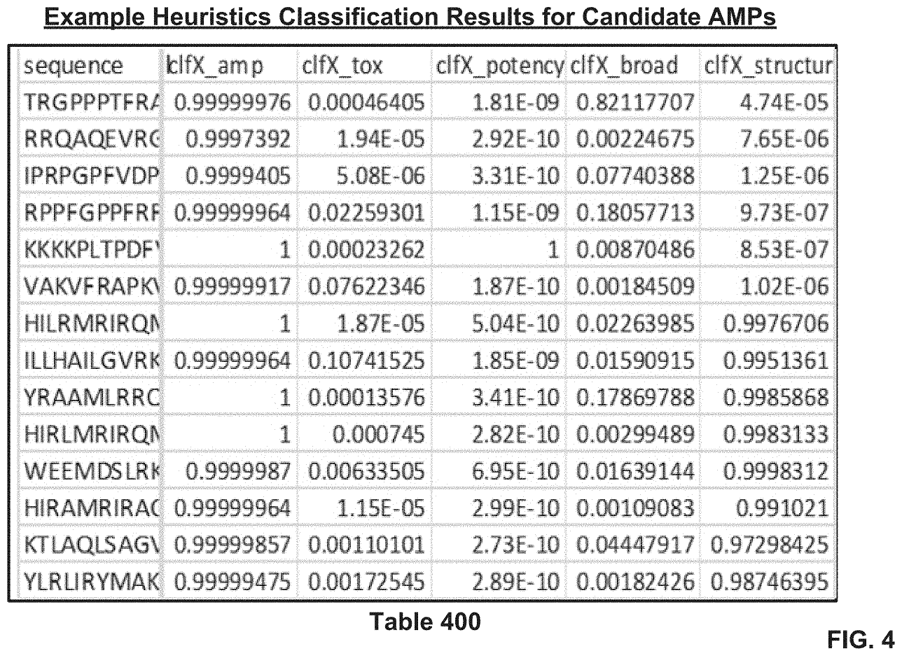

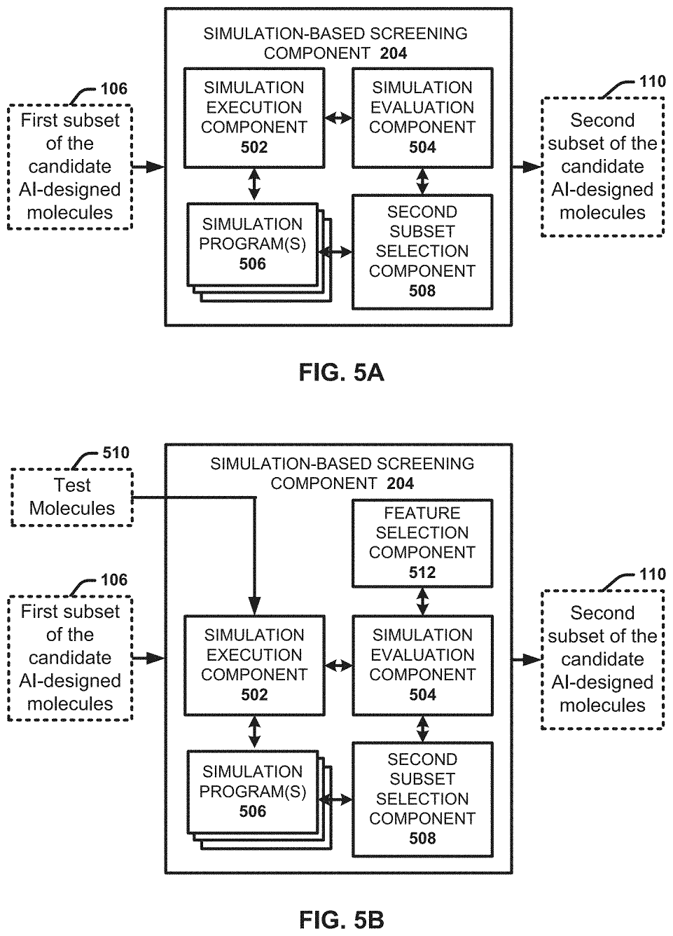

Regardless of the distribution of AI-designed molecules included in the initial set 102, the heuristics-based screening component 202 and the simulation-based screening component 204 can be configured to screen the candidates based on a target biological activity/function and/or target chemical activity/function. For example, in implementations in which the target biological activity/function is providing broad spectrum antimicrobial activity (e.g., activity against both Gram positive and Gram negative strains), the heuristics-based screening component 202 and the simulation-based screening component 204 can be configured to screen the candidates to select a small subset (e.g., the second subset 110 of the candidate AI-designed molecules) of the most viable candidates that are expected to provide broad spectrum antimicrobial activity. Additional details of the heuristics-based screening component 202 are described with reference to FIGS. 3A and 3B and FIG. 4. Additional details of the simulation-based screening component 204 are described with reference to FIGS. 5A-9.

FIGS. 3A and 3B illustrates block diagrams of example heuristics-based screening components in accordance with one or more embodiments. Repetitive description of like elements employed in respective embodiments is omitted for sake of brevity.

In accordance with the embodiment shown in FIG. 3A, the heuristics-based screening component 202 can include classifier application component 302, first subset selection component 304 and one or more classifiers 306. In various embodiments, the classifier application component 302 can be configured to apply the one or more classifiers to the initial set 102 of candidate AI-designed molecules to determine or infer whether each (or in some implementations one or more) of the initial candidate molecules has one or more of the defined target features (i.e., features of interest) based on analysis of their respective molecular sequences (e.g., protein sequence, genetic/nucleotide sequence, polymer sequence, and the like) and/or their chemical structures. In this regard, the heuristic-based screening phase is based on analysis and classification of the candidate molecules at the sequence-level and/or chemical structure level.

The one or more defined target features can be preselected and reflect one or more desired features for the target AI-designed molecules that disclosed filtering techniques are being used to identify. The one or more features can include explicit features (e.g., exhibits antimicrobial activity, exhibits broad spectrum susceptibility), as well as implicit features that have a known correlation to the explicit features (e.g., having a secondary peptide structure which has been correlated to antimicrobial activity). The one or more target features can thus vary based on the specific application of pipeline 100 and/or system 200.

For example, in some embodiments, pipeline 100 and/or system 200 can be applied to screen candidate AI-designed peptides to identify and select a small subset of the candidate AI-designed peptides that are the most likely to effective, provide broad-spectrum antimicrobial agents. With these embodiments, the one or more defined features can include (but are not limited to), antimicrobial functionality, broad-spectrum efficacy, low or no toxicity, potency, and presence a defined structure (e.g., a secondary structure such as a helix structure, a pleated sheet structure, a coil structure, etc.). The one or more classifiers 306 can thus be configured to predict whether each of the initial candidate peptides have antimicrobial functionality (or not), have broad-spectrum efficacy (or not), have low or no toxicity (or not), have defined secondary structure (or not), and/or have high potency or not.

In some embodiments, the one or more classifiers 306 can include one or more binary classification models that have been previously trained to classify the respective candidates as either having or not having the one or more defined target features based on learned correlations between the defined target features and patterns reflected in molecular sequences (e.g., protein sequences) and/or chemical structures of known molecules that have the target features. In other implementations, the one or more classifiers 306 can be configured to predict probabilities that the candidate molecules have the respective target features (e.g., probability of having target feature 1, probability of having target feature 2, probability of having target feature 3, etc.) In some implementations, each classifier of the one or more classifiers 306 can be trained to classify a single target feature. For example, with respect to the AMP implementation described above, the one or more classifiers 306 can include up to four separate classifiers, one for each of the four target features (e.g., antimicrobial functionality, broad-spectrum efficacy, low or no toxicity, and presence a defined structure).

Various types of classification models/algorithms can be used for the one or more classifiers 306. In some embodiments, the one or more classifiers 306 can include one or more deep neural network-based classifiers, such as a long short-term memory (LSTM) neural network-based classifier. The heuristics-based screening component 202 can also employ an automatic classification system and/or an automatic classification process to facilitate classifying one or more target features of the initial candidate molecules. For example, the heuristics-based screening component can employ a probabilistic and/or statistical-based analysis (e.g., factoring into the analysis utilities and costs) to learn and/or generate inferences with respect to the initial set 102 of candidate AI-designed molecules. The heuristics-based screening component 202 can employ, for example, a support vector machine (SVM) classifier to learn and/or generate inferences for initial set 102 of candidates.

Additionally, or alternatively, the one or more classifiers 306 can employ classification techniques associated with Bayesian networks, decision trees and/or probabilistic classification models. The one or more classifiers 306 can also include explicitly trained (e.g., via a generic training data) as well as implicitly trained (e.g., via receiving extrinsic information) classifiers. For example, with respect to SVM's, SVM's can be configured via a learning or training phase within a classifier constructor and feature selection module. In some implementations, the one or more classifiers 306 can also include non-binary classifiers that map an input attribute vector, x=(x1, x2, x3, x4, xn), to a confidence that the input belongs to a class—that is, f(x)=confidence(class). With these implementations, the classifier application component 302 can determine a measure of confidence in the predictions that the candidates have or do not have each of the evaluated target features.

The first subset selection component 304 can be configured to select the first subset 106 of the candidate AI-designed molecules from the initial set 102 based on the classification results and defined selection criterial. The selection criteria can be predefined, adjusted by the system administrator, and the like. For example, in some implementations, the selection criteria can require the first subset selection component 304 to select only those candidates that are determined to have (or classified as having) all of the defined target features. In another example, the selection criteria can require the first subset selection component 304 to select those candidates that are determined to have (or classified as having) one or more of the defined target features. In another example, the selection criteria can require the first subset selection component 304 to select those candidates that are determined to have (or classified as having) specific combinations of target features have one or more of the defined target features. In another example, in implementations in which the one or more classifiers 306 determine values representative of the probabilities that a candidate molecule has the respective probabilities, the selection criteria can include defined thresholds for the probabilities and/or scores representative of the collective probabilities for all the features.

It should be appreciated that the selection criteria can be tailored as appropriate for a particular application (e.g., with respect to number of defined features required, combinations of features required, values indicative of a level of exhibition of the features, values indicative of degree of confidence in the classification inferences, etc.).

FIG. 3B presents another embodiment of the heuristics-based screening component 202. In the embodiment shown in FIG. 3B, the heuristics-based screening component 202 further includes classifier training component 308 to facilitate training and developing the one or more classifiers 306. With these embodiments, the classifier training component 308 can employ one or more unsupervised, supervised, and/or semi-supervised machine learning techniques to train and develop the one or more classifiers 306 based on received or otherwise available training data 310. For example, the training data 310 can include a plurality of molecular sequences (e.g., protein sequences) whose classification with respect to one or more of the target features is known, including sequences with positive classifications (e.g., that have one or more particular target features) and negative classifications (e.g., that do not have one or more particular target features). Using sets of positive and negative sequences for each target feature, the classifier training component 308 can train a separate classifier for each target feature.

FIG. 4 provides a table 400 presenting example heuristics classification results for candidate antimicrobial peptides (AMPs) in accordance with one or more embodiments. In particular, Table 400 presents example heuristics classification data that can be generated and/or determined by the classifier application component 302 based on application of five different classifiers to a plurality of candidate AMP sequences based on their respective peptide sequences shown in the first column. The five different classifiers are respectively identified with notation “clfX_feature”, wherein “clr is an acronym and the “X” indicates the particular training data set used to train the classifiers.

The first classifier, clfX._amp (wherein “amp” represents” antimicrobial peptide”) determined the probability (from 0.0 to 1.0) that the peptide sequences have antimicrobial activity (or otherwise are AMPs). The second classifier, clfX._tox (wherein “tox” represents “toxicity”) determined the probability (from 0.0 to 1.0) that the peptide sequences are toxic. The third classifier, clfX._potency determined the probability (from 0.0 to 1.0) that the peptide sequences are potent. The fourth classifier, clfX._broad (wherein the “broad” represents “broad spectrum”) determined the probability (from 0.0 to 1.0) that the peptide sequences are broad-spectrum antimicrobials. The fifth classifier, clfX._structur (wherein “structur” represents “structure” determined the probability (from 0.0 to 1.0) that the peptide sequences have a secondary structure.

FIGS. 5A and 5B illustrates block diagrams of example simulation-based screening components in accordance with one or more embodiments. Repetitive description of like elements employed in respective embodiments is omitted for sake of brevity.

The simulation-based screening component 204 provides for further refining the first subset 106 of the AI-designed molecules into an even smaller, second subset 110 of the candidate AI-designed molecules to recommend for wet laboratory testing using a high-throughput, computationally efficient, and physically-inspired filtering process that uses physics-based molecular computer simulations. These computer simulations simulate the molecular interactions between respective candidates included in the first subset 106 and one or more known or potential molecular and/or biological targets (e.g., one or more cellular components of a pathogen) to determine whether and/or to what degree the simulated candidates exhibit one or more desired interaction characteristics. In this regard, the one or more desired interactions (or desired behavioral characteristics) can include one or more predefined and/or learned interaction behaviors/characteristics that are correlated with achieving the target biological/molecular activity, function or response (e.g., antimicrobial activity, antiviral activity, a specific therapeutic activity, etc.). For example, in implementations in which the target biological/molecular activity/response includes being an effective antimicrobial agent, the one or more desired interactions/behavioral characteristics can include one or more molecular interaction behavioral characteristics that are correlated with exterminating bacteria and/or inhibiting bacterial growth.

With reference to FIG. 5A, to facilitate this end, the simulation-based screening component 204 can include simulation execution component 502, simulation evaluation component 504 one or more simulation programs 506, and second subset selection component 508.

The one or more simulation programs 506 can include the one or more high-throughput computer simulation programs that can simulate physics-based molecular interactions. In particular, the one or more simulation programs 506 can provide molecular simulation tools capable of simulating molecular interactions between AI-designed molecules and one or more biological/molecular targets based on their modeled molecular and/or biological structures. For example, these simulation tools can include course-grained molecular dynamics (CGMD) simulation tools, and the like. For example, in some implementations, the one or more simulation programs 506 can include receive and/or generate molecular models for the respective candidate molecules included in the first subset 106. In some implementations, the molecular models can include all-atom models. The one or more simulation programs 506 can further receive and/or generate a molecular model for the biological/molecular target(s) (e.g., one or more cellular components of a pathogen) modeled as a forcefield (e.g., a course-grained forcefield or the like). The one or more simulation programs 506 can further generate course-grained system representations for combinations of the molecular candidates and the biological/molecular target(s) (e.g., one or more cellular components of a pathogen) and employ the course-grained system representations to simulate the molecular dynamics of the interactions between the respective candidates and the biological/molecular target(s).

The simulation execution component 502 can be configured to execute/run the one or more simulations on respective candidates included in the first subset 106. In this regard, the simulation execution component 502 can run a CGMD for each (or in some implementations one or more) candidate AI-designed molecule included in the first subset 106, wherein each simulation simulates the molecular interactions between each candidate molecule and one or more defined biological/molecular targets based on their respective modeled molecular structures as modeled using one or more forcefield models.

The simulation evaluation component 504 can be configured to evaluate the respective simulations to determine whether and/or to what degree each candidate AI-designed molecule simulated (i.e., each candidate molecule included in the first subset 106) exhibits the one or more target molecular interactions/behavioral characteristics. For example, in some implementations, the molecular simulation program used can be configured to identify and track occurrence of the one or more target molecular interactions/behavioral characteristics over the course of each simulation. With these embodiments, the simulation program can generate results data for each simulation that indicates whether the one or more target molecular interactions/behavioral characteristics occurred, frequency of occurrence, and the like. The simulation evaluation component 504 can further employ the results data generated for each simulation to determine whether and/or to what degree each candidate AI-designed molecule simulated (i.e., each candidate molecule included in the first subset 106) exhibits the one or more target molecular interactions/behavioral characteristics. In other embodiments, the simulations can be manually observed and evaluated to determine whether and/or to what degree each candidate AI-designed molecule simulated exhibits the one or more target molecular interactions/behavioral characteristics. With these embodiments, such results data can be received as user generated feedback.

The second subset selection component 508 can further select one or more of the simulated candidate molecules for inclusion in the second subset 110 based on whether and/or to what degree the one or more simulated candidate molecules exhibit the one or more target molecular interactions/behavioral characteristics. For example, in some implementations, the second subset selection component 508 can be configured to select any of the simulated candidates that are determined to exhibit the one or more target molecular interactions/behavioral characteristics. In other implementations, the second subset selection component 508 can be configured to select one or more of the simulated candidates that are determined to exhibit the one or more target molecular interactions/behavioral characteristics with consistent and/or sufficient propensity (e.g., relative to a defined threshold valuation for measuring consistent and/or sufficient propensity). In another example implementation, the second subset selection component 508 can be configured to select one or more of the simulated candidates that are determined to “best” exhibit the one or more target molecular interactions/behavioral characteristics, as measured using a defined valuation scheme. In this regard, the valuation scheme and the selection criteria can vary based on the types of molecular interactions/behaviors evaluated and the manner in which they can be measured.

In one or more exemplary embodiments in which the candidates AI-designed molecules are candidate AMPs, to screen whether the candidate peptides are promising antimicrobials, the simulation execution component 502 can run computer simulations (e.g., CGMD simulations or the like) of the interaction between each of the candidate peptides included in the first subset 106 with a model lipid bilayer or another cellular component of a pathogen. The lipid bilayer can consist of a mixture of lipids. For example, the candidate peptides can be modeled with a suitable all-atom representation of the peptide given its protein sequence (e.g., prepared as an alpha helix or a s random coil). The model lipid bilayer can further be modelled using a forcefield model (e.g., a coarse-grained forcefield model or the like). The modeled peptide structures can further be transformed into course-grained representations and combined with the membrane model to create a course-grained peptide-membrane system for simulation.

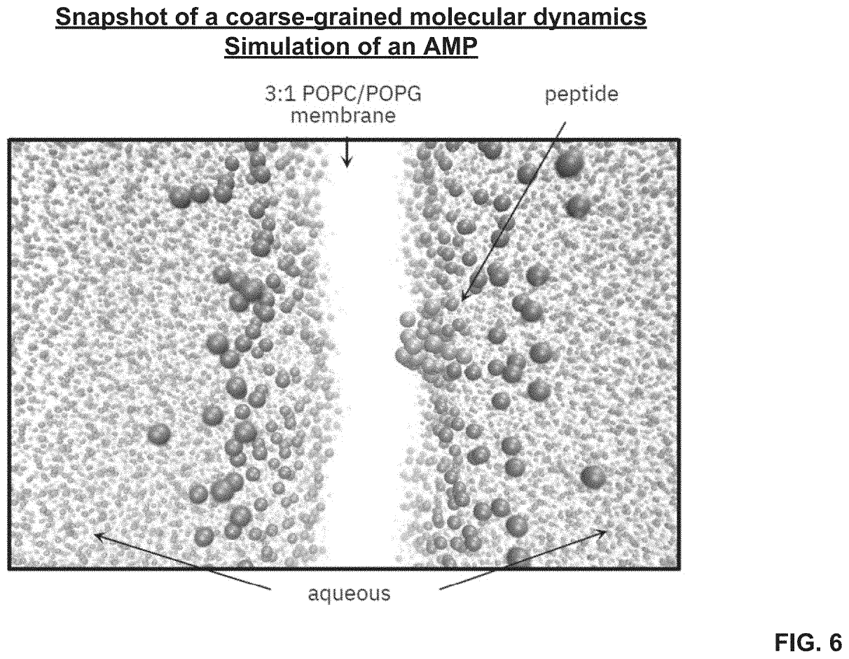

For example, FIG. 6 provides a snapshot of a course-grained molecular dynamics simulation of an AMP in accordance with one or more embodiments. In this simulation the modeled peptide is bound to the modeled lipid bilayer, which in this example simulation is a 3:1 mixture of phosphatidylcholine (POPC) and palmitoyloleoyl PG (POPG). FIG. 6 depicts a CGMD simulation using the modeled peptides and the modeled membrane. In accordance with these simulations, the respective candidate peptides are interacted with the membrane for 1.0 microsecond (μ). The physical dynamics of the interaction are then evaluated to determine whether the interactions indicate the peptides indicate the provide antimicrobial activity.

In one or more embodiments, the target interactions/behaviors used to evaluate antimicrobial propensity based on the above described computer simulations can be based on the number of contacts/touch points between the peptide and the membrane and the stability of those contacts. In this regard, as described in greater detail with reference to FIG. 5B, antimicrobial propensity was found to strongly correlate with the number of contacts and the contact stability, wherein the greater the number of contacts and the greater stability of those contacts, the greater probability of antimicrobial propensity. The contacts can include contacts between the positive residues of the peptide and the membrane. In one or more implementations, the number of contacts between positive residues and the lipid membranes is defined as the number of atoms belonging to a lipid at a distance less than 7.5 Å from a positive residue of the peptide. Contact stability can be measured as a function of the variance in the number of contacts, wherein the lower the variance the greater the stability and thus the higher indication of strong antimicrobial activity.

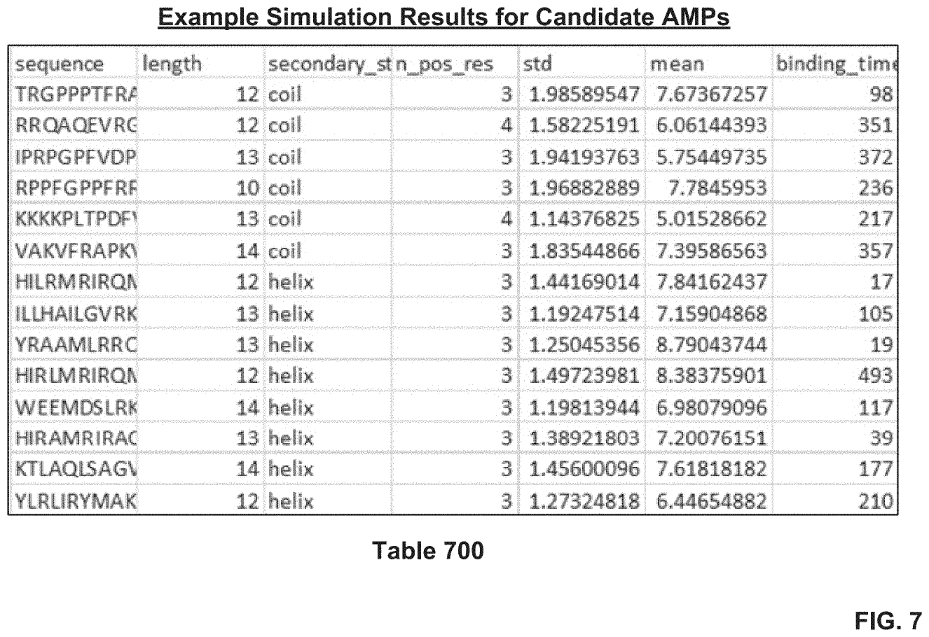

FIG. 7 provides a table 700 presenting example simulation results for candidate AMPs in accordance with one or more embodiments. Table 700 provides example computer simulation results for a plurality of example candidate peptide sequences, respectively identified in the first column. The peptide length, their respective secondary structures and the number of positive residues for each sequence are respectively included in the second, third and fourth column. The fifth column provides the standard deviation (std) of the number of contacts, which corresponds to the variance of the number of contacts. The sixth column provides the mean of the number of contacts. The seventh column provides the binding time in nanoseconds (ns). The binding time represents the duration of time the peptide took to form the contacts following initiation of the simulation. In the embodiment shown, all example peptides formed their contacts in less than 500 (ns), (which is preferable and can also be used as a filtering criteria).

With reference again to FIG. 5A in view of FIG. 7, in furtherance to the AMP candidate screening embodiments, the simulation evaluation component 504 can determine and/or receive simulation results (such as those provided in table 700) that identifies the number of contacts and the variance of the number of contacts between the lipids and the positive residues of for each of the candidate peptides. In some implementations, the simulation results can also include the binding time, which can further be used as a filtering criterion, as noted above. The second subset selection component 508 can further select one or more of the candidate peptides that exhibit consistent membrane interaction propensity, as determined based on the number of contacts, the variance values, and/or the binding time. For example, in one or more embodiments, the second subset selection component 508 can employ defined variance acceptability criteria and select only those candidate peptides whose variance values, number of contacts, and/or binding time satisfy defined acceptability criteria. In some implementations, the defined acceptability criteria can require the variance value (i.e., the standard deviation) to be 2.0 beads or less, the number of contacts to be 5.0 or more (averaged over the duration of the simulation), and whose binding time is less than 500 ns during the 1.0 us long simulation time (e.g., so that the contact variance is calculated over at least half of the total simulation time).

With now to FIG. 5B presented is another example of the simulation-based screening component 204 in accordance with one or more additional embodiments. Repetitive description of like elements employed in respective embodiments is omitted for sake of brevity.

In the embodiments described above directed to simulation-based screening of candidate AMPs, the example, target molecular interaction features/behaviors that we evaluated and used to select the second subset of the candidate AI-designed molecules included number of contacts/touch points between the peptide and the membrane and the stability of those contacts (as measured in variance in the number of contacts). These target features were discovered by running test simulations using the same molecular modeling simulations described above as applied to known peptide sequences known to have antimicrobial activity and known peptide sequences known to lack antimicrobial activity, since there exists no standardized protocol for screening antimicrobial candidates using molecular simulations.

Based on analysis of the results of the test runs for both the positive and negative antimicrobial peptides, the specific target features described above were identified for the first time. In this regard, the test simulation runs demonstrated that that the variance of the number of contacts between positive residues and membrane lipids is predictive of antimicrobial activity.

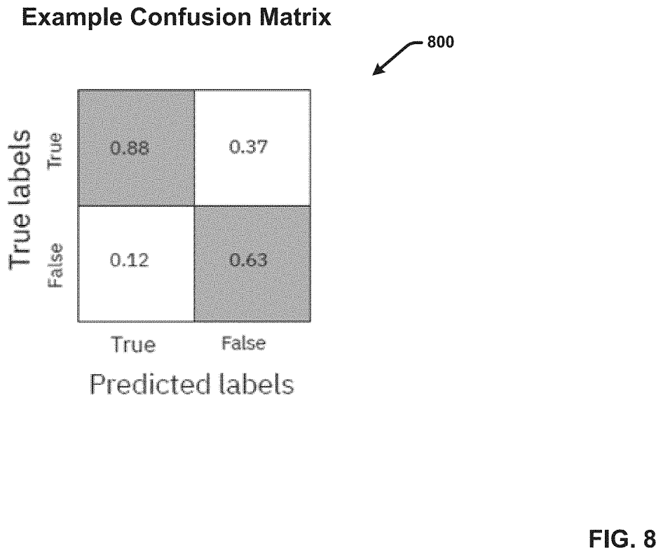

In particular, FIG. 8 presents an example confusion matrix 600 of the simulation-based classifier that uses peptide-membrane contact variance as the feature for detecting viable AMP sequences. The confusion matrix 600 demonstrates that we can predict the antimicrobials with 88% accuracy by using features contact variance features that were derived from the above described simulations alone. Specifically, the contact variance distinguishes between high potency and non-antimicrobial sequences with a sensitivity of 88% and a specificity of 63%. Physically, this feature can be interpreted as measuring the robust binding tendency of a sequence to model membrane.

In various embodiments, this test simulation process can be performed and/or facilitated by the simulation-based screening component 204 using the simulation execution component 502 and the feature selection component 512. This test simulation process can also be applied to determine the target features for the simulation screening process as applied to other types of AI-designed molecules for a variety of different target biological activities.

In this regard, in some embodiments, training high-throughput computer simulations can be performed for test molecules including test molecules that are known to be effective at achieving the target activity of the AI-designed molecules (e.g., the desired biological activity in implementations in which the AI-designed molecules are pharmaceuticals) and optionally molecules that are known to be ineffective, to identify the one or more behavioral characteristics that correlate with effectiveness in achieving the target activity. These one or more behavioral characteristics can be used as the one or more target characteristics that are used to evaluate (e.g., by the simulation evaluation component 504) and select (e.g., by the second subset selection component 508) the second subset 110 of candidates when the computer simulations are run on the unknown sequences of the candidates.

With these embodiments, the simulation execution component 502 can receive (or otherwise access) test molecules 510 that correspond to the initial set of candidate AI molecules or more specifically, that correspond to the first subset of candidate AI-designed molecules whose target biological activity status is known (e.g. antimicrobial activity/inactivity status). In this regard, the test molecules 510 can include both molecules known to provide the target biological activity and molecules known to not provide the target biological activity. The simulation execution component 502 can further be configured to apply the same computer simulations (e.g., provided by the simulation programs 506) that will be used on the first subset 106 to the test molecules 510. The simulations on the test molecules can further be evaluated to identify one or more target features/or characteristics that correlate to the target biological activity desired to be provided by the AI-designed molecules being evaluated (e.g., antimicrobial activity, antiviral activity, etc.). For example, with respect to the AMR simulation embodiments described above, the selected features included the variance in the number of contacts. Once identified, these features can then be used to classify them based on the target feature (e.g., the number of contacts between the lipids and the positive residues of the peptide) and select the second subset 110 of candidates for laboratory testing.

In the embodiment in FIG. 5B, the simulation-based screening component 204 can further include feature selection component 512 to facilitate identified these target features based on analysis of the test simulations for the positive and negative test molecules. In this regard, the feature selection component 512 can employ one or more machine learning techniques to identify target features/or characteristics that correlate to the target biological activity desired to be provided by the AI-designed molecules being evaluated (e.g., antimicrobial activity, antiviral activity, etc.) based on correlations and patterns in the test simulation data. The machine learning techniques can include supervised machine learning techniques, semi-supervised machine learning techniques, unsupervised machine learning techniques, or a combination thereof. For example, the machine learning techniques can include usage of the various classification techniques described herein, as well as expert systems, fuzzy logic, SVMs, Hidden Markov Models (HMMs), greedy search algorithms, rule-based systems, Bayesian models (e.g., Bayesian networks), neural networks, other non-linear training techniques, data fusion, utility-based analytical systems, systems employing Bayesian models, and the like.

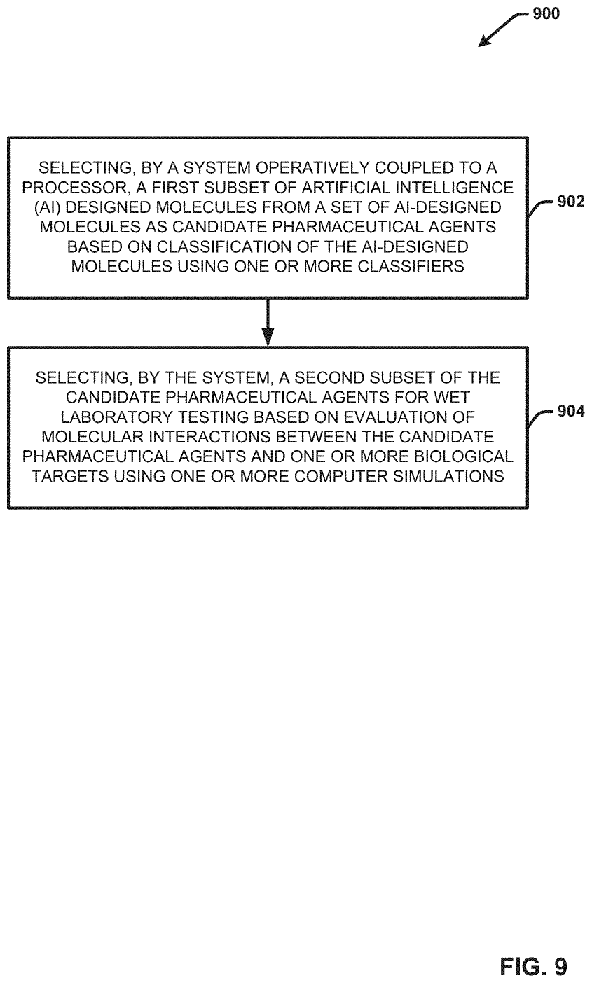

FIG. 9 illustrates a high-level flow diagram of an example, non-limiting computer-implemented method 900 for filtering AI-designed molecules for laboratory testing in accordance with one or more embodiments. Repetitive description of like elements employed in respective embodiments are omitted for sake of brevity.

At 902, a system operatively coupled to a processor (e.g., system 200 or the like) selecting, by a system operatively coupled to a processor, a first subset of artificial intelligence (AI) designed molecules from a set of AI-designed molecules as candidate pharmaceutical agents based on classification of the AI-designed molecules using one or more classifiers (e.g., using the heuristics-based screening component 202). At 904 the system selects a second subset of the candidate pharmaceutical agents for wet laboratory testing based on evaluation of molecular interactions between the candidate pharmaceutical agents and one or more biological targets (e.g., one or more cellular components of a pathogen) using one or more computer simulations (e.g., using the simulation-based screening component 204).

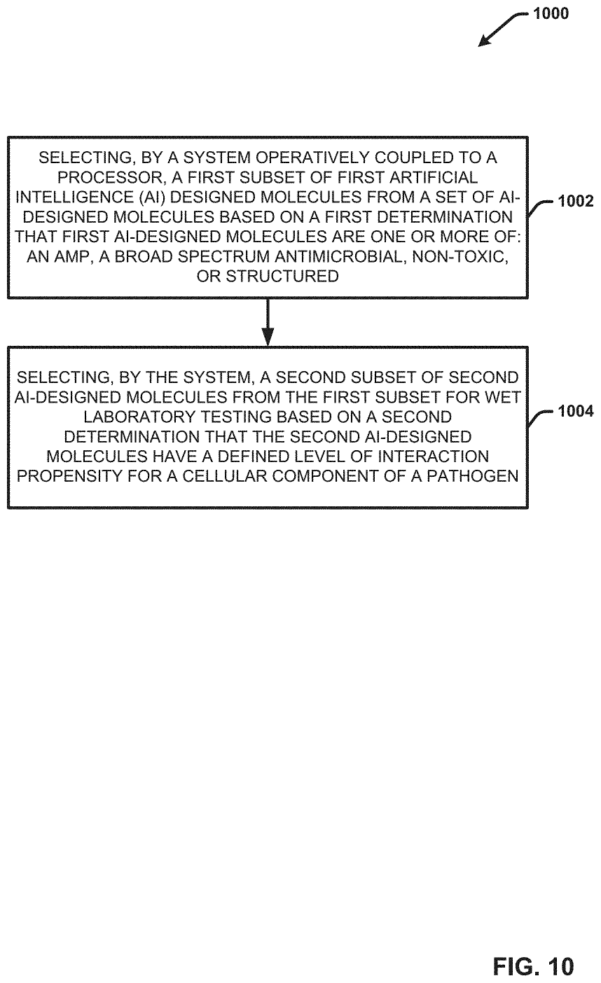

FIG. 10 illustrates a high-level flow diagram of an example, non-limiting computer-implemented method 1000 for filtering candidate AI-designed antimicrobial molecules for laboratory testing in accordance with one or more embodiments. Repetitive description of like elements employed in respective embodiments are omitted for sake of brevity.

At 1002, a system operatively coupled to a processor (e.g., system 200 or the like) can select a first subset of first artificial intelligence (AI) designed molecules from a set of AI-designed molecules based on a first determination that first AI-designed molecules are one or more of: an AMP, a broad spectrum antimicrobial, non-toxic, or structured (e.g., using the heuristics-based screening component 202). For example, in one or more embodiments the heuristics-based screening component 202 can employ one or more trained classifiers to determine whether each (or in some implementations one or more) of the candidate AI-designed molecules included in the initial set are an AMP or not, broad-spectrum or not, toxic or not, and/or structured or not, as described above with reference to FIG. 3A, FIG. 3B, and FIG. 4. At 1004, the system can select a second subset of second AI-designed molecules from the first subset for wet laboratory testing based on a second determination that the second AI-designed molecules have a defined level of interaction propensity for a cellular component of a pathogen (e.g., using the simulation-based screening component 204). For example, in one or more embodiments, as described above with reference to FIGS. 5A-8, the simulation-based screening component 204 can employ one or more computer simulations of the molecular dynamics for each of the candidate peptides included in the first subset relative to a modeled cellular component of a pathogen (e.g., a lipid bilayer or another cellular component) to determine their interaction propensity as a function of contact variance.

The screening techniques described herein have proven successful when applied to screen thousands of AI-designed AMPs to identify viable candidates. In particular, the disclosed screening techniques where applied to an initial set of about 100,000 candidate peptides generated using an AI-based peptide design method referred to as Conditional Latent (attribute) Space Sampling, or CLaSS. The CLaSS design method employs an attribute conditioned/controlled sampling from an informative latent space learned using a neural generative model to generate candidate AMPs.

The initial set of 100,000 candidate peptides was reduced to 163 candidate peptides using the heuristic-based screening process. To screen the initial 100,000 CLaSS-generated AMP sequences for experimental validation, an independent set of four binary (yes/no) sequence-level deep neural net-based classifiers were used to predict antimicrobial function, broad-spectrum efficacy (e.g., activity on both Gram positive and Gram negative strains), presence of secondary structure, as well as toxicity, in accordance with the heuristics-based screening process described above. A bidirectional LSTM-based classifier was trained for each of the four attributes on a labeled training dataset for known peptide sequences with a hidden layer size of 100 and a dropout of 0.3. Based on the distribution of the scores (classification probabilities/logits), the threshold was determined by considering the 50^{th} percentile (median) of the scores. The screening criteria used to select the first subset of candidates from the initial 100,000 viable candidates thus considered all four attributes. 163 candidates passed this screening.

The 163 candidate peptides were then subjected to coarse-grained Molecular Dynamics (CGMD) simulations of peptide-membrane interactions to test for membrane-binding tendency in accordance with the simulation-based screening process described above. The simulation-based screening resulted in identification of 20 lead candidate peptides that exhibited high and consistent membrane-binding activity in the computer simulations. These top 20 peptides have the following sequences: YLRLIRYMAKMI (SEQ ID NO: 1), FPLTWLKWWKWKK (SEQ ID NO: 2), HILRMRIRQMMT (SEQ ID NO: 3), ILLHAILGVRKKL (SEQ ID NO: 4), YRAAMLRRQYMMT (SEQ ID NO: 5), HIRLMRIRQMMT (SEQ ID NO: 6), HIRAMRIRAQMMT (SEQ ID NO: 7), KTLAQLSAGVKRWH (SEQ ID NO: 8), HILRMRIRQGMMT (SEQ ID NO: 9), HRAIMLRIRQMMT (SEQ ID NO: 10), EYLIEVRESAKMTQ (SEQ ID NO: 11), GLITMLKVGLAKVQ (SEQ ID NO: 12), YQLLRIMRINIA (SEQ ID NO: 13), VRWIEYWREKWRT (SEQ ID NO: 14), LIQVAPLGRLLKRR (SEQ ID NO: 15), YQLRLIMKYAI (SEQ ID NO: 16), HRALMRIRQCMT (SEQ ID NO: 17), GWLPTEKWRKLC (SEQ ID NO: 18), YQLRLMRIMSRI (SEQ ID NO: 19), LRPAFKVSK (SEQ ID NO: 20), and conservatively modified variants thereof.

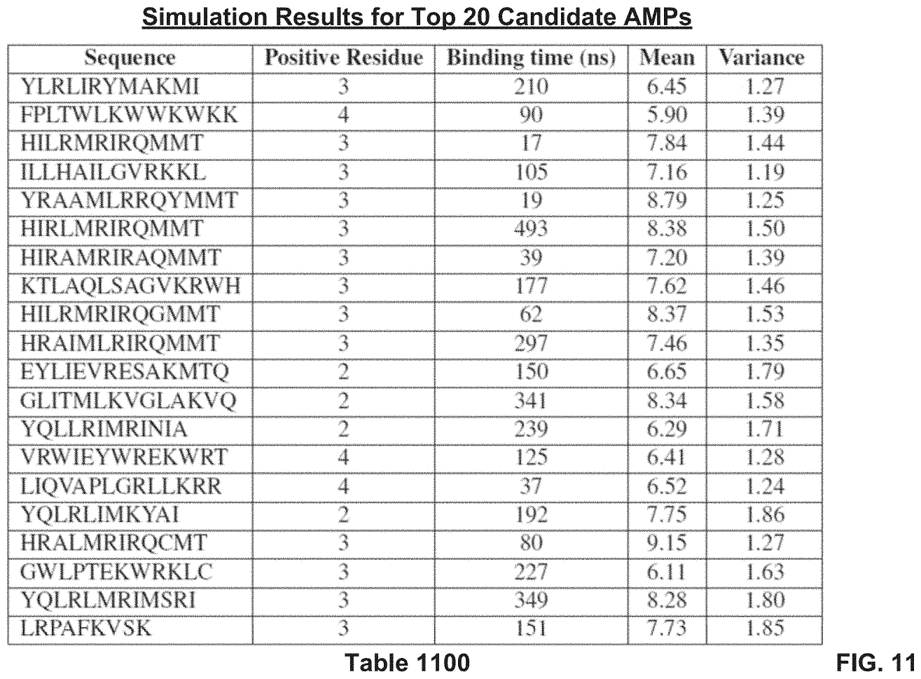

FIG. 11 provides a table 1100 presenting the simulation results for the top 20 CLaSS-generated AMPs selected from the 163 candidate peptides selected after the heuristic-based screening process. Table 1100 presents the physics-derived features of the simulation-based screening, such as mean and variance of the number of contacts between positive amino acids and membrane beads (that are found to be associated with antimicrobial function), as extracted from CGMD simulations of peptide membrane interactions. The criteria employed to further filter the 163 candidates required the variance value (i.e., the standard deviation) to be 2.0 beads or less, the number of contacts to be 5.0 or more (averaged over the duration of the simulation), and the binding time to be less than 500 ns during the 1.0 us long simulation time. Based on the combination of the CLaSS generation method, the ML heuristic-based screening process and the molecular simulation results, these top 20 peptides demonstrate strong antimicrobial activity or behaviour and are thus promising broad spectrum antimicrobial agents. These top 20 peptides are further characterized as having low toxicity.

The 20 lead candidate peptides were then synthesized and tested using wet laboratory experiments for antimicrobial activity and toxicity. Among these 20 lead peptides two novel AMPs with the highest antimicrobial activity were identified. These two novel AMPs were experimentally validated with strong broad-spectrum anti-microbial activity and low in vitro and in vivo toxicity. Both of the novel AMPs were not present in the supervised training data used to design the initial candidate CLaSS peptides. These experiments demonstrate that the disclosed three-stage screening pipeline for AI-generated AMP sequences (e.g., ML heuristic screening, simulation screening, and wet laboratory screening) yields a success rate of 1 out of 10 at the final stage.

It should be noted that, for simplicity of explanation, in some circumstances the computer-implemented methodologies are depicted and described herein as a series of acts. It is to be understood and appreciated that the subject innovation is not limited by the acts illustrated and/or by the order of acts, for example acts can occur in various orders and/or concurrently, and with other acts not presented and described herein. Furthermore, not all illustrated acts can be required to implement the computer-implemented methodologies in accordance with the disclosed subject matter. In addition, those skilled in the art will understand and appreciate that the computer-implemented methodologies could alternatively be represented as a series of interrelated states via a state diagram or events. Additionally, it should be further appreciated that the computer-implemented methodologies disclosed hereinafter and throughout this specification are capable of being stored on an article of manufacture to facilitate transporting and transferring such computer-implemented methodologies to computers. The term article of manufacture, as used herein, is intended to encompass a computer program accessible from any computer-readable device or storage media.

FIG. 12 can provide a non-limiting context for the various aspects of the disclosed subject matter, intended to provide a general description of a suitable environment in which the various aspects of the disclosed subject matter can be implemented. FIG. 12 illustrates a block diagram of an example, non-limiting operating environment in which one or more embodiments described herein can be facilitated. Repetitive description of like elements employed in other embodiments described herein is omitted for sake of brevity.

With reference to FIG. 12, a suitable operating environment 1200 for implementing various aspects of this disclosure can also include a computer 1212. The computer 1212 can also include a processing unit 1216, a system memory 1214, and a system bus 1218. The system bus 1218 couples system components including, but not limited to, the system memory 1214 to the processing unit 1216. The processing unit 1216 can be any of various available processors. Dual microprocessors and other multiprocessor architectures also can be employed as the processing unit 1216. The system bus 1218 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MCA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Firewire (IEEE 1294), and Small Computer Systems Interface (SCSI).

The system memory 1214 can also include volatile memory 1220 and nonvolatile memory 1222. The basic input/output system (BIOS), containing the basic routines to transfer information between elements within the computer 1212, such as during start-up, is stored in nonvolatile memory 1222. Computer 1212 can also include removable/non-removable, volatile/non-volatile computer storage media. FIG. 12 illustrates, for example, a disk storage 1224. Disk storage 1224 can also include, but is not limited to, devices like a magnetic disk drive, floppy disk drive, tape drive, Jaz drive, Zip drive, LS-100 drive, flash memory card, or memory stick. The disk storage 1224 also can include storage media separately or in combination with other storage media. To facilitate connection of the disk storage 1224 to the system bus 1218, a removable or non-removable interface is typically used, such as interface 1226. FIG. 12 also depicts software that acts as an intermediary between users and the basic computer resources described in the suitable operating environment 1200. Such software can also include, for example, an operating system 1228. Operating system 1228, which can be stored on disk storage 1224, acts to control and allocate resources of the computer 1212.

System applications 1230 take advantage of the management of resources by operating system 1228 through program modules 1232 and program data 1234, e.g., stored either in system memory 1214 or on disk storage 1224. It is to be appreciated that this disclosure can be implemented with various operating systems or combinations of operating systems. A user enters commands or information into the computer 1212 through input device(s) 1236. Input devices 1236 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1216 through the system bus 1218 via interface port(s) 1238. Interface port(s) 1238 include, for example, a serial port, a parallel port, a game port, and a universal serial bus (USB). Output device(s) 1240 use some of the same type of ports as input device(s) 1236. Thus, for example, a USB port can be used to provide input to computer 1212, and to output information from computer 1212 to an output device 1240. Output adapter 1242 is provided to illustrate that there are some output devices 1240 like monitors, speakers, and printers, among other output devices 1240, which require special adapters. The output adapters 1242 include, by way of illustration and not limitation, video and sound cards that provide a means of connection between the output device 1240 and the system bus 1218. It should be noted that other devices and/or systems of devices provide both input and output capabilities such as remote computer(s) 1244.

Computer 1212 can operate in a networked environment using logical connections to one or more remote computers, such as remote computer(s) 1244. The remote computer(s) 1244 can be a computer, a server, a router, a network PC, a workstation, a microprocessor based appliance, a peer device or other common network node and the like, and typically can also include many or all of the elements described relative to computer 1212. For purposes of brevity, only a memory storage device 1246 is illustrated with remote computer(s) 1244. Remote computer(s) 1244 is logically connected to computer 1212 through a network interface 1248 and then physically connected via communication connection 1250. Network interface 1248 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL). Communication connection(s) 1250 refers to the hardware/software employed to connect the network interface 1248 to the system bus 1218. While communication connection 1250 is shown for illustrative clarity inside computer 1212, it can also be external to computer 1212. The hardware/software for connection to the network interface 1248 can also include, for exemplary purposes only, internal and external technologies such as, modems including regular telephone grade modems, cable modems and DSL modems, ISDN adapters, and Ethernet cards.

One or more embodiments described herein can be a system, a method, an apparatus and/or a computer program product at any possible technical detail level of integration. The computer program product can include a computer readable storage medium (or media) having computer readable program instructions thereon for causing a processor to carry out aspects of one or more embodiment. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. In this regard, in various embodiments, a computer readable storage medium as used herein can include non-transitory and tangible computer readable storage mediums.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of one or more embodiments can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of one or more embodiments.

Aspects of one or more embodiments are described herein with reference to flowchart illustrations and/or block diagrams of methods, apparatus (systems), and computer program products according to embodiments. It will be understood that each block of the flowchart illustrations and/or block diagrams, and combinations of blocks in the flowchart illustrations and/or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and other devices to function in a particular manner, such that the computer readable storage medium having instructions stored therein comprises an article of manufacture including instructions which implement aspects of the function/act specified in the flowchart and block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart and block diagram block or blocks.

The flowchart and block diagrams in the Figures illustrate the architecture, functionality, and operation of possible implementations of systems, methods, and computer program products according to various embodiments described herein. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and flowchart illustration, and combinations of blocks in the block diagrams and flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on one or more computers, those skilled in the art will recognize that this disclosure also can or can be implemented in combination with other program modules. Generally, program modules include routines, programs, components, data structures, etc. that perform particular tasks or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices. For example, in one or more embodiments, computer executable components can be executed from memory that can include or be comprised of one or more distributed memory units. As used herein, the term “memory” and “memory unit” are interchangeable. Further, one or more embodiments described herein can execute code of the computer executable components in a distributed manner, e.g., multiple processors combining or working cooperatively to execute code from one or more distributed memory units. As used herein, the term “memory” can encompass a single memory or memory unit at one location or multiple memories or memory units at one or more locations.

As used in this application, the terms “component,” “system,” “platform,” “interface,” and the like, can refer to and can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and a computer. By way of illustration, both an application running on a server and the server can be a component. One or more components can reside within a process or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that can provide specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

The term “facilitate” as used herein is in the context of a system, device or component “facilitating” one or more actions or operations, in respect of the nature of complex computing environments in which multiple components and/or multiple devices can be involved in some computing operations. Non-limiting examples of actions that may or may not involve multiple components and/or multiple devices comprise transmitting or receiving data, establishing a connection between devices, determining intermediate results toward obtaining a result (e.g., including employing machine learning and artificial intelligence to determine the intermediate results), etc. In this regard, a computing device or component can facilitate an operation by playing any part in accomplishing the operation. When operations of a component are described herein, it is thus to be understood that where the operations are described as facilitated by the component, the operations can be optionally completed with the cooperation of one or more other computing devices or components, such as, but not limited to: sensors, antennae, audio and/or visual output devices, other devices, etc.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches, and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
a memory that stores computer executable components;
a processor that executes the computer executable components stored in the memory, wherein the computer executable components comprise:
a heuristics-based screening component that evaluates a set of artificial intelligence (AI) designed molecules using one or more classifiers to select a first subset of the AI-designed molecules as candidate pharmaceutical agents; and
a simulation-based screening component that evaluates the candidate pharmaceutical agents using one or more computer simulations of molecular interactions between the candidate pharmaceutical agents and one or more biological targets to select a second subset of the candidate pharmaceutical agents for wet laboratory testing.

2. The system of claim 1, wherein the one or more classifiers comprise one or more machine learning models that classify the AI-designed molecules as having or not having one or more defined features of a target pharmaceutical agent based on molecular sequences of the AI-designed molecules.

3. The system of claim 2, wherein the heuristics-based screening component selects the first subset based on the first subset having the one or more defined features.

4. The system of claim 1, wherein the one or more computer simulations employ one or more force field models for the candidate pharmaceutical agents and the one or more biological targets.

5. The system of claim 1, wherein the simulation-based screening component selects the second subset based on the second subset exhibiting one or more target molecular interaction features in the one or more computer simulations.

6. The system of claim 1, wherein the candidate pharmaceutical agents comprise candidate antimicrobial agents, and wherein the one or more classifiers determine whether the AI-designed molecules are at least one of: an antimicrobial peptide, a broad-spectrum antimicrobial, non-toxic, or structured.

7. The system of claim 6, wherein the simulation-based screening component employs the one or more computer simulations to evaluate interaction propensity between the candidate antimicrobial agents and a model lipid bilayer comprising, or another cellular component of a pathogen, and a forcefield.

8. The system of claim 7, wherein the simulation-based screening component selects the second subset of the candidate antimicrobial agents for laboratory testing based on the second subset exhibiting a defined level of the interaction propensity.

9. The system of claim 6, wherein the simulation-based screening component employs initial computer simulations to simulate interactions between test molecules having potent and inactive sequences with a model lipid bilayer, or another cellular component of a pathogen, and selects one or more features correlate with antimicrobial activity based on the interactions.

10. The system of claim 9, wherein the simulation-based screening component evaluates the candidate antimicrobial agents for inclusion in the second subset based on whether the candidate antimicrobial agents exhibit the one or more features as determined using the one or more computer simulations.

11. The system of claim 6, wherein the wet laboratory testing comprises at least one of:
testing the second subset against one or more pathogens, including gram-positive bacteria and gram-negative bacteria; or
testing a toxicity of the second subset.

12. A method, comprising:
selecting, by a system operatively coupled to a processor, a first subset of artificial intelligence (AI) designed molecules from a set of AI-designed molecules as candidate pharmaceutical agents based on classification of the AI-designed molecules using one or more classifiers; and
selecting, by the system, a second subset of the candidate pharmaceutical agents for wet laboratory testing based on evaluation of molecular interactions between the candidate pharmaceutical agents and one or more biological targets using one or more computer simulations.

13. The method of claim 12, wherein the one or more classifiers comprise one or more machine learning models that classify the AI-designed molecules as having or not having one or more defined features of a target pharmaceutical agent based on molecular sequences of the AI-designed molecules.

14. The method of claim 13, wherein the selecting the first subset comprises selecting the first subset based on the first subset having the one or more defined features.

15. The method of claim 12, wherein the selecting the second subset comprises selecting the second subset based on the second subset exhibiting one or more target molecular interaction features in the one or more computer simulations.

16. The method of claim 12, wherein the candidate pharmaceutical agents comprise candidate antimicrobial agents, and wherein the classification comprises determining, by the system, whether the AI-designed molecules comprise one or more features selected from the group consisting of: antimicrobial functionality, broad-spectrum efficacy, non-toxic, and presence a defined secondary structure.

17. The method of claim 16, wherein the method further comprises:
employing, by the system, the one or more computer simulations to evaluate interaction propensity between the candidate antimicrobial agents and a model lipid bilayer comprising or another cellular component of a pathogen and a forcefield, wherein the selecting the second subset comprises selecting the second subset based on the second subset exhibiting a defined level of the interaction propensity.

18. The method of claim 16, further comprising:
employing, by the system, initial computer simulations to evaluate interactions between test proteins having potent and inactive sequences with a model lipid bilayer or another cellular component of a pathogen and a forcefield;
selecting, by the system, one or more features derived from the interactions that correlate with antimicrobial activity; and
evaluating, by the system, the candidate antimicrobial agents for inclusion in the second subset based on whether the candidate antimicrobial agents exhibit the one or more features as determined using the one or more computer simulations.

19. The method of claim 16, wherein the wet laboratory testing comprises at least one of:
testing the second subset against one or more pathogens, including gram-positive bacteria and gram-negative bacteria; or
testing the toxicity of the second subset.

20. A computer program product for filtering and validating artificial intelligence (AI)-designed molecules, the computer program product comprising a computer readable storage medium having program instructions embodied therewith, the program instructions executable by a processing component to cause the processing component to:
select a first subset of the AI-designed molecules from as candidate pharmaceutical agents based on classification of the AI-designed molecules using one or more classifiers; and
select a second subset of the candidate pharmaceutical agents for wet laboratory testing based on evaluation of molecular interactions between the candidate pharmaceutical agents and one or more biological targets using one or more computer simulations.


SEQUENCE LISTING (20 sequences)

SEQ ID NO: 1 - protein, 12 aa, Artificial Sequence
  synthetic antimicrobial peptide designed using artificial intelligence
YLRLIRYMAKMI

SEQ ID NO: 2 - protein, 13 aa, Artificial Sequence
  synthetic antimicrobial peptide designed using artificial intelligence
FPLTWLKWWKWKK

SEQ ID NO: 3 - protein, 12 aa, Artificial Sequence
  synthetic antimicrobial peptide designed using artificial intelligence
HILRMRIRQMMT

SEQ ID NO: 4 - protein, 13 aa, Artificial Sequence
  synthetic antimicrobial peptide designed using artificial intelligence
ILLHAILGVRKKL

SEQ ID NO: 5 - protein, 13 aa, Artificial Sequence
  synthetic antimicrobial peptide designed using artificial intelligence
YRAAMLRRQYMMT

SEQ ID NO: 6 - protein, 12 aa, Artificial Sequence
  synthetic antimicrobial peptide designed using artificial intelligence
HIRLMRIRQMMT

SEQ ID NO: 7 - protein, 13 aa, Artificial Sequence
  synthetic antimicrobial peptide designed using artificial intelligence
HIRAMRIRAQMMT

SEQ ID NO: 8 - protein, 14 aa, Artificial Sequence
  synthetic antimicrobial peptide designed using artificial intelligence
KTLAQLSAGVKRWH

SEQ ID NO: 9 - protein, 13 aa, Artificial Sequence
  synthetic antimicrobial peptide designed using artificial intelligence
HILRMRIRQGMMT

SEQ ID NO: 10 - protein, 13 aa, Artificial Sequence
  synthetic antimicrobial peptide designed using artificial intelligence
HRAIMLRIRQMMT

SEQ ID NO: 11 - protein, 14 aa, Artificial Sequence
  synthetic antimicrobial peptide designed using artificial intelligence
EYLIEVRESAKMTQ

SEQ ID NO: 12 - protein, 14 aa, Artificial Sequence
  synthetic antimicrobial peptide designed using artificial intelligence
GLITMLKVGLAKVQ

SEQ ID NO: 13 - protein, 12 aa, Artificial Sequence
  synthetic antimicrobial peptide designed using artificial intelligence
YQLLRIMRINIA

SEQ ID NO: 14 - protein, 13 aa, Artificial Sequence
  synthetic antimicrobial peptide designed using artificial intelligence
VRWIEYWREKWRT

SEQ ID NO: 15 - protein, 14 aa, Artificial Sequence
  synthetic antimicrobial peptide designed using artificial intelligence
LIQVAPLGRLLKRR

SEQ ID NO: 16 - protein, 11 aa, Artificial Sequence
  synthetic antimicrobial peptide designed using artificial intelligence
YQLRLIMKYAI

SEQ ID NO: 17 - protein, 12 aa, Artificial Sequence
  synthetic antimicrobial peptide designed using artificial intelligence
HRALMRIRQCMT

SEQ ID NO: 18 - protein, 12 aa, Artificial Sequence
  synthetic antimicrobial peptide designed using artificial intelligence
GWLPTEKWRKLC

SEQ ID NO: 19 - protein, 12 aa, Artificial Sequence
  synthetic antimicrobial peptide designed using artificial intelligence
YQLRLMRIMSRI

SEQ ID NO: 20 - protein, 9 aa, Artificial Sequence
  synthetic antimicrobial peptide designed using artificial intelligence
LRPAFKVSK